(19) United States
(12) Patent Application Publication
(10) Pub. No.: US 2026/0256477 A1
(43) Pub. Date: Sep. 3, 2026

(54) SURGICAL DRILLING DEVICES AND METHODS OF USE

(71) Applicants: Smith & Nephew, Inc., Memphis, TN (US); Smith & Nephew Orthopaedics AG, Zug (CH); Smith & Nephew Asia Pacific PTE. Limited, Singpore (SG)

(72) Inventors: Dennis COLLERAN, Boston, MA (US); Rick Fu, Haverhill, MA (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 19/656,443

(22) Filed: Apr. 23, 2026

Related U.S. Application Data
(63) Continuation of application No. PCT/US2024/051405, filed on Oct. 15, 2024.
(60) Provisional application No. 63/652,422, filed on May 28, 2024.
(60) Provisional application No. 63/593,299, filed on Oct. 26, 2023.

(51) Int. Cl. A61B 17/16; A61B 17/00

(52) U.S. Cl. CPC A61B 17/1617; A61B 17/1622; A61B 2017/00367

(57) ABSTRACT

A surgical drilling device includes a drill bit at a distal end of a shaft configured to drill a bone hole having a first diameter. A cutter disposed within the shaft can move between a closed position and an extended position. The cutter is configured to drill a bone hole having a second diameter in the extended position. An adjustable member is at least partially disposed in the handle. An actuator is operatively coupled to both the adjustable member and the cutter. A distal end of the actuator has an arcuate projection configured to engage an arcuate slot in the cutter. Incremental movement of the adjustable member causes a corresponding incremental degree of extension of the cutter between the closed position and the extended position to drill the bone hole having the second diameter.

### CROSS-REFERENCED TO RELATED APPLICATIONS

This application is a continuation of International Application No. PCT/US2024/051405, filed on October 15, 2024, entitled SURGICAL DRILLING DEVICES AND METHODS OF USE, which in turn claims priority to and benefit of U.S. Provisional Application No. 63/593,299, filed on October 26, 2023, and U.S. Provisional Application No. 63/652,422, filed on May 28, 2024. The entire contents of the above-listed applications are incorporated herein by reference in their entirety.

### FIELD

The present disclosure relates generally to surgical drilling devices and, more specifically, to surgical drilling devices for creating tunnels through bone during arthroscopic ligament reconstruction surgery.

### BACKGROUND

Desired outcomes for arthroscopic ligament reconstruction surgery are generally achieved by establishing the proper shape and placement of torn tissue. While performing such surgery, a surgeon typically makes a small incision in a patient’s skin covering the surgical site (e.g., a bone joint) to allow a surgical device to be placed in the bone joint and manipulated through arthroscopic visualization. In some arthroscopic procedures, such as anterior cruciate ligament (ACL) and posterior cruciate ligament (PCL) reconstruction, it is often desirable to create a bone tunnel of varying diameters to match a given graft size. A portion of this tunnel may accept a large graft, while the remainder may accept only a suture and an implant to secure that graft. Current techniques vary significantly and may use a variety of tools, including fixed diameter drills, drills that have a smaller entry diameter and then adjust to a single larger diameter, and drills that have a smaller entry diameter and then adjust to multiple larger diameters. However, such drills typically can only drill the larger diameter in a retrograde direction.

### SUMMARY

The disclosure describes an adjustable drill that has a small core diameter (i.e., about 3.5 mm) and can expand to drill holes from 3.5 to 12 mm in diameter. A surgeon can insert the drill through a small portal or cannula, adjust the cutting area by a dial to a larger diameter in the joint, and create the larger diameter bone tunnel in either an antegrade or retrograde direction. Advantageously, the drill of this disclosure allows the surgeon to place one tool and accomplish all necessary drilling with that one tool, simplifying and streamlining the technique.

Further examples of the surgical drilling devices and methods of this disclosure may include one or more of the following, in any suitable combination.

In examples, a surgical drilling device of this disclosure includes a shaft extending through a handle. The shaft has a proximal end, a distal end, and a longitudinal axis extending therebetween. The distal end of the shaft has a drill bit configured to drill a bone hole having a first diameter. A cutter is disposed within the shaft. The cutter is configured to move between a closed position, in which the cutter is fully contained within the shaft, and an extended position, in which the cutter at least partially extends from a sidewall of the shaft. The cutter is configured to drill a bone hole having a second diameter different from the first diameter. An adjustable member is at least partially arranged within the handle. An actuator is disposed within the shaft and operatively coupled to both the adjustable member and the cutter. A distal end of the actuator includes an arcuate projection configured to engage an arcuate slot in the cutter. Incremental movement of the adjustable member causes a corresponding incremental degree of extension of the cutter between the closed position and the extended position to drill the bone hole having the second diameter.

In further examples, the first diameter is 3.5 mm. In examples, the second diameter is between 4 mm and 12 mm. In examples, the adjustable member is a rotatable dial. In examples, the rotatable dial is configured to rotate in a direction of the longitudinal axis. In examples, a retainer is configured to hold the rotatable dial in a desired rotational position. In examples, the retainer is one of a spring plunger, a wave spring, a ball plunger, and a flex beam. In examples, the rotatable dial includes a central opening and a cam track arranged at least partially around the central opening. The cam track has a spiral formation such that a distance between a position of the cam track and the central opening varies radially. In examples, the actuator is operatively coupled to the cam track of the adjustable member by a sliding pin moveable through a slot defined in the actuator. In examples, the sliding pin is configured to travel along the cam track as a user rotates the adjustable member. In examples, the cutter is configured to extend through a slot defined in the sidewall of the shaft. In examples, an outer surface of the shaft comprises visual scale markings to indicate a penetration depth of the surgical drilling device into a bone. In examples, the cutter is pivotably connected to the distal end of the shaft by a single pivot pin. In examples, the adjustable member has a plurality of visual scale markings corresponding to either of the first or the second diameter. In examples, the cutter is configured to drill the bone hole having the second diameter in both an antegrade and a retrograde direction.

In examples, a method of drilling a bone hole of this disclosure includes placing a surgical drilling device adjacent a bone. The surgical drilling device includes a shaft extending through a handle. The shaft has a proximal end, a distal end, and a longitudinal axis extending therebetween. The distal end of the shaft includes a drill bit. A cutter is disposed within the shaft. The cutter is configured to move between a closed position, in which the cutter is fully contained within the shaft, and an extended position, in which the cutter at least partially extends from a sidewall of the shaft. An adjustable member is at least partially arranged within the handle. An actuator is disposed within the shaft and operatively coupled to both the adjustable member and the cutter. A distal end of the actuator includes an arcuate projection configured to engage an arcuate slot in the cutter. Using the drill bit, a bone hole having a first diameter is drilled. The adjustable member is moved to cause the cutter to move to the extended position. Using the cutter, a bone hole having a second diameter different from the first diameter is drilled. Incremental movement of the adjustable member causes a corresponding incremental degree of extension of the cutter between the closed position and the extended position to drill the bone hole having the second diameter.

In further examples, the first diameter is 3.5 mm. In examples, the second diameter is between 4 mm and 12 mm. In examples, moving the adjustable member includes rotating the adjustable member in a direction of the longitudinal axis. In examples, drilling the bone hole having the second diameter includes drilling in either an antegrade or a retrograde direction.

A reading of the following detailed description and a review of the associated drawings will make apparent the advantages of these and other structures. Both the foregoing general description and the following detailed description serve as an explanation only and do not restrict aspects of the disclosure as claimed.

### BRIEF DESCRIPTION OF THE DRAWINGS

The disclosure will be more fully understood by reference to the detailed description, in conjunction with the following figures, wherein:

FIG. 1A illustrates an assembled view of a surgical drilling device, according to some embodiments of the disclosure;

FIGS. 1B and 1C illustrate a detailed view of the distal end of the shaft of the surgical drilling device, according to some embodiments of the disclosure;

FIG. 1D illustrates an exploded view of the surgical drilling device, according to some embodiments of the disclosure;

FIGS. 2A-2C illustrate the operation of the adjustable member of the surgical drilling device, according to some embodiments of the disclosure;

FIGS. 3A-3D illustrate the operation of the cutter of the device, according to some embodiments of the disclosure; and

FIGS. 4A-4D illustrate examples of a retainer of the surgical drilling device, according to some embodiments of the disclosure.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

As used in the specification and claims, for the purposes of describing and defining the invention, the terms “about” and “substantially” are used to represent the inherent degree of uncertainty that may be attributed to any quantitative comparison, value, measurement, or other representation. The terms “about” and “substantially” are also used herein to represent the degree by which a quantitative representation may vary from a stated reference without resulting in a change in the basic function of the subject matter at issue. “Comprise,” “include,” and/or plural forms of each are open ended and include the listed parts and can include additional parts that are not listed. “And/or” is open-ended and includes one or more of the listed parts and combinations of the listed parts.

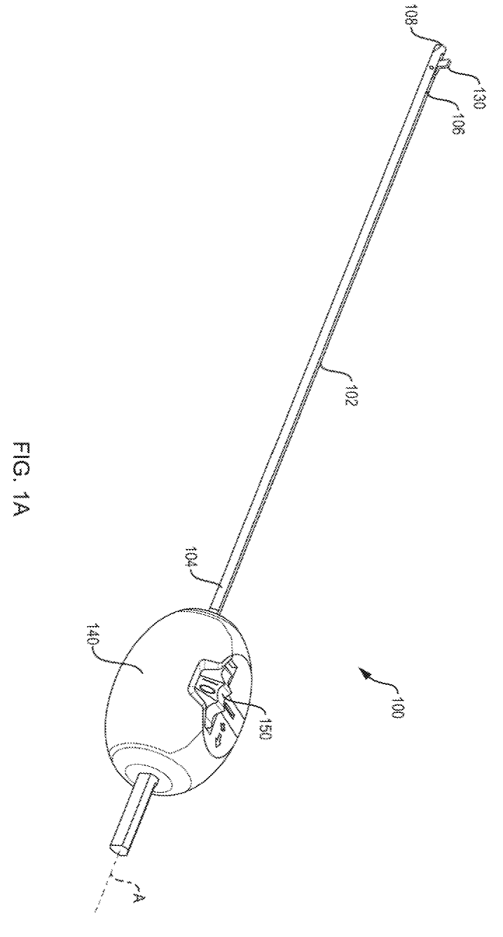

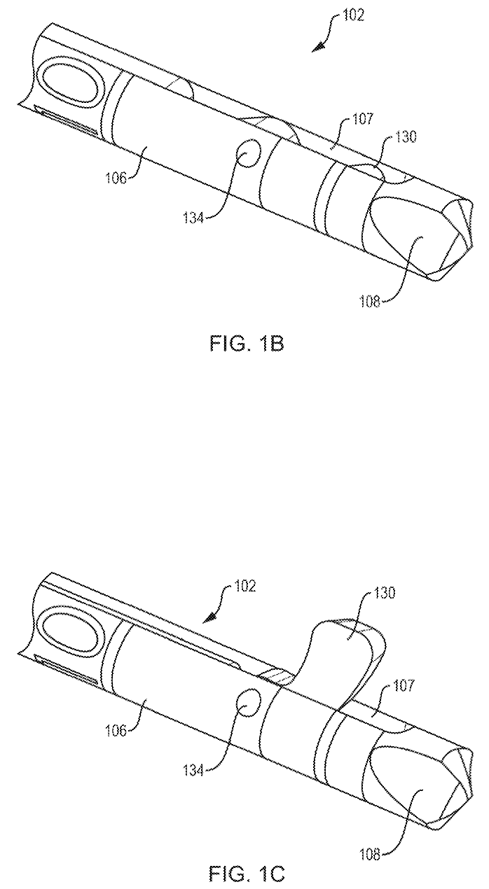

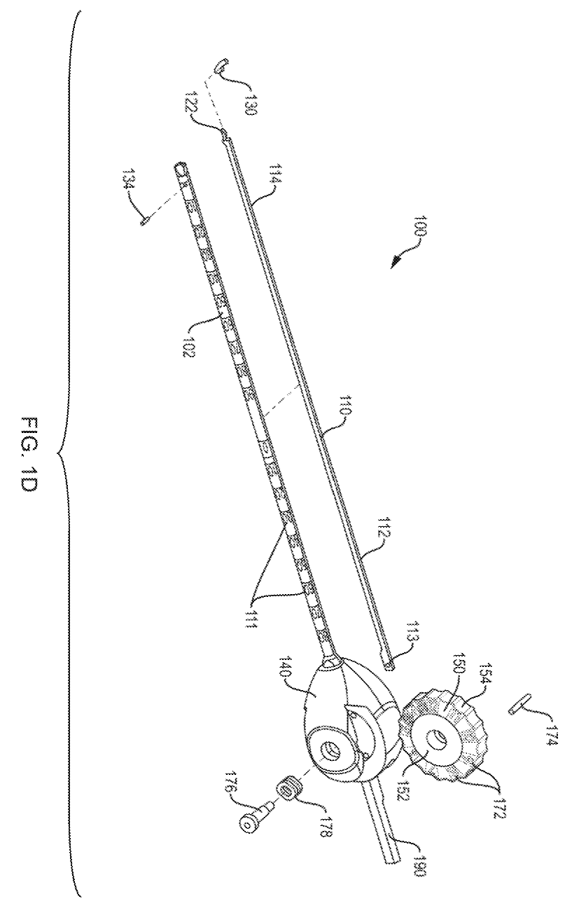

FIG. 1A illustrates an assembled view of a surgical drilling device 100, according to some embodiments of the disclosure. As shown in FIG. 1A, the device 100 may generally include a handle 140 and a shaft 102 extending from the handle 140. The shaft 102 may have a proximal end 104 and a distal end 106, and may be arranged along a longitudinal axis A. The distal end 106 of the shaft 102 may include a drill bit 108 for drilling a bone hole in an antegrade direction. An adjustable member 150 (e.g., a rotatable dial) may be at least partially arranged within the handle 140 and be operatively connected to a cutter 130 disposed at the distal end 106 of the shaft 102. The adjustable member 150 may be rotatable by the surgeon in the direction of the longitudinal axis A to enable opening of the cutter 130 to various radial extents. Advantageously, the cutter 130 may have a shape that allows the cutter 130 to be deployable close to the distal end 106 of the shaft 102. This allows the cutter 130 to be deployed in very tight spaces without damaging adjacent anatomic structures of the patient.

FIGS. 1B and 1C illustrate detailed views of the distal end 106 of the shaft 102. As shown in FIGS. 1B and 1C, the distal end 106 of the shaft 102 may include the drill bit 108 configured for drilling a hole in bone or other tissue, such that the bone hole has a first diameter (e.g., 3.5 mm). The cutter 130 may be disposed within the shaft 102 proximal to the drill bit 108 and may be pivotably connected to the shaft 102 by a single pivot pin 134. The cutter 130 may be configured to move between a closed position, in which the cutter 130 is fully contained within the shaft 102 (FIG. 1B), and an open position, in which the cutter 130 at least partially extends from a sidewall of the shaft 102 (FIG. 1C). For example, the cutter 130 may extend through a slot 107 defined by a sidewall of the shaft 102 to different radial extents. As such, the cutter 130 may be used to drill or overdrill bone holes having various diameters (e.g., 4 mm to 12 mm) in both an antegrade and retrograde direction.

FIG. 1D illustrates an exploded view of the device 100, according to some embodiments of the disclosure. As shown in FIG. 1D, an outer surface of the shaft 102 may include visual scale markings 111 to enable the surgeon to identify a penetration depth of the device 100 into the patient’s bone. An actuator 110 having a proximal end 112 and a distal end 114 may be adapted to be coaxially received within the shaft 102. The distal end 114 of the actuator 110 may be configured to be operatively connected to the cutter 130, for example, by means of an arcuate projection 122, as described in more detail below. The proximal end 112 of the actuator 110 may be operatively connected to the adjustable member 150 such that the actuator 110 is moveable relative to the shaft 102 by movement of the adjustable member 150. For example, a sliding pin 174 may extend through an opening 113 in the proximal end 114 of the actuator 110 to couple the actuator 110 to the adjustable member 150, as further described below. The adjustable member 150 may include visual scale markings 172 about a circumference of the adjustable member 150 to indicate the desired bone hole diameter to be drilled within the patient’s bone using the cutter 130. The visual scale markings 172 may be provided on opposing sidewalls 152 of the adjustable member 150 near a rim gripping surface 154. A center of the adjustable member 150 may be fixedly attached to the handle 140 by means of a center pin 176 and a washer 178. A bit connector 190 may be adapted to be attached to a power tool. The power tool may be configured to impart rotational movement to the device 100. While FIG. 1D illustrates the adjustable member 150 as a rotatable dial, the disclosure also contemplates other types of adjustable members 150. For example, the adjustable member 150 could be a threaded ring displaceable along the shaft 102, thereby moving the actuator 110 and deploying the cutter 130 to the desired diameter.

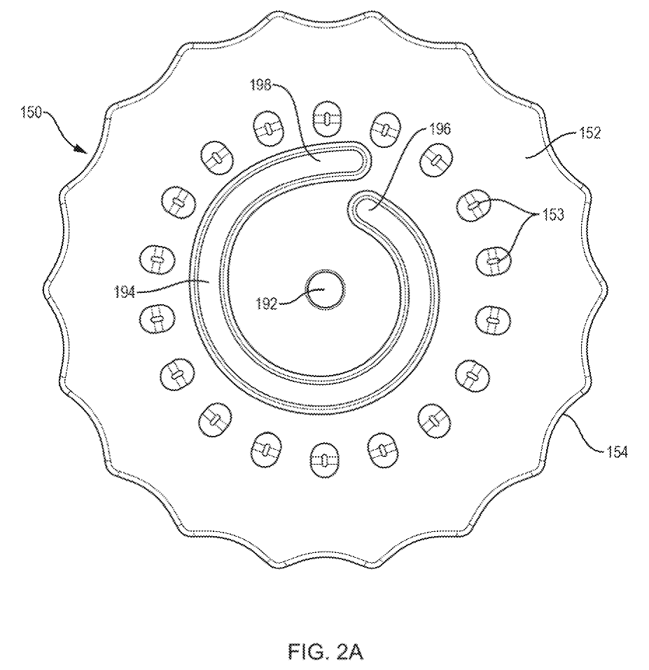

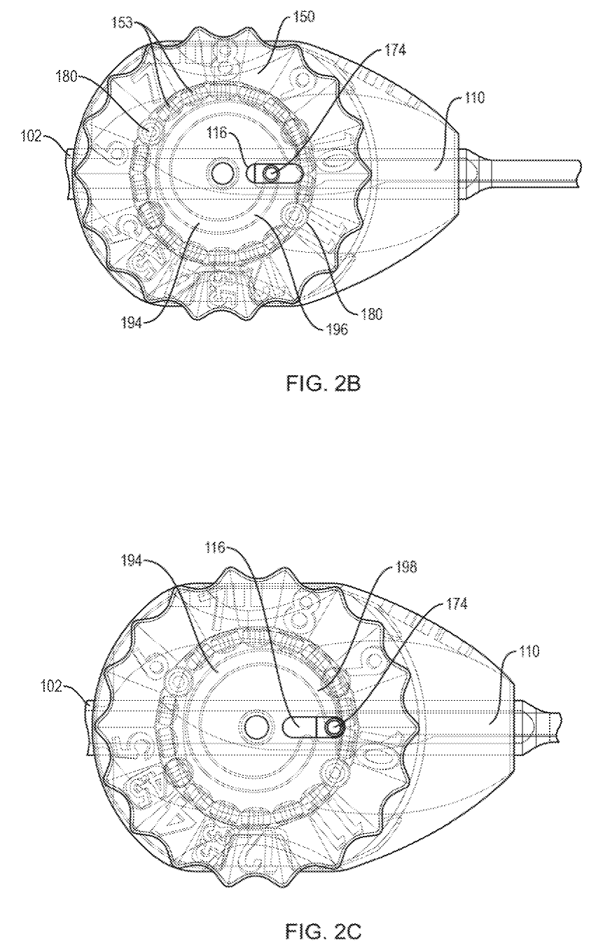

FIGS. 2A-2C illustrate the cooperation between the adjustable member 150 and the actuator 110, according to some embodiments of the disclosure.

As shown in FIG. 2A, the adjustable member 150 may comprise opposing sidewalls 152 and the circumferential gripping surface 154. The gripping surface 154 may be corrugated or otherwise textured to facilitate a surgeon’s finger interaction with the adjustable member 150. A central opening 192 may be formed at a center of the adjustable member 150 and may be sized and shaped to receive the center pin 176 and the washer 178 (FIG. 1D). A cam track 194 may be formed through the adjustable member 150 and may be arranged at least partially around the central opening 192. The cam track 194 may include a first end 196 and a second end 198. The cam track 194 may have a spiral formation such that a distance between a position of the cam track 194 and the central opening 192 varies radially to correspond to linear movement of the actuator 110, as described further below. A plurality of recesses 153 may be formed on at least one of the sidewalls 152 and arranged at least partially around central opening 192.

As shown in FIG. 2B and 2C, the actuator 110 may be moveably coupled to the adjustable member 150, for example, by means of the sliding pin 174 moveable through a slot 116 defined in the actuator 110. The sliding pin 174 may also be configured to travel along the cam track 194 as a surgeon rotates the adjustable member 150. Rotation of the adjustable member 150 by the surgeon may cause longitudinal displacement of the sliding pin 174 through the slot 116 as it moves along the cam track 194, in turn causing linear displacement of the actuator 110 through the shaft 102. FIG. 2B shows the relative positions of the actuator 110 and the adjustable member 150 when the cutter 130 is in the fully closed position. In this position, the sliding pin 174 may be positioned at the first end 196 of the cam track 194 and closer to a proximal end of the slot 116. At least one retainer 180 may further engage at least one of the recesses 153 to hold the adjustable member 150 in the desired rotational position. FIG. 2C shows the relative position of the actuator 110 and the adjustable member 150 when the cutter 130 is in the fully opened (e.g., 12 mm) position. In this position, the sliding pin 174 may be located at the second end 198 of the cam track 194 and closer to a distal end of the slot 116. However, incremental rotation of the adjustable member 150 (e.g., in 0.5 mm increments) may cause a corresponding incremental displacement of the sliding pin 174, and hence the actuator 110. Incremental displacement of the actuator 110 may in turn define the extent of the radial extension of the cutter 130 with respect to the outer circumference of the shaft 102.

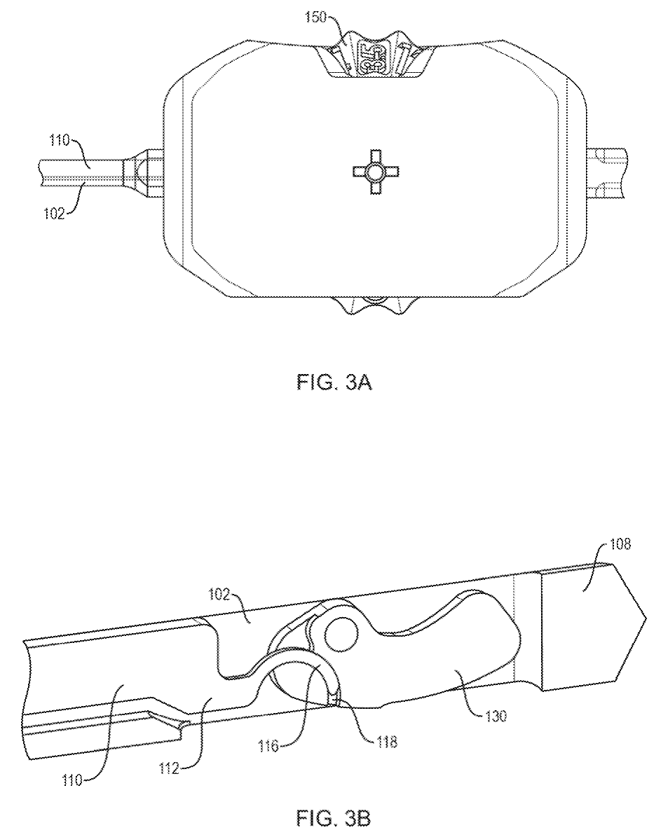

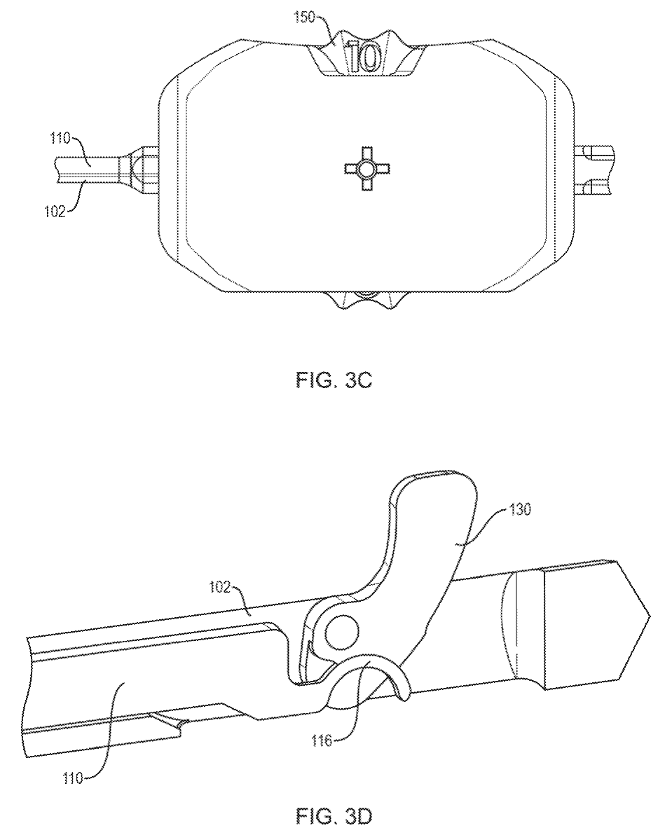

FIGS. 3A-3D illustrate the operation of the cutter 130 of the device 100, according to some embodiments of the disclosure.

FIG. 3A illustrates the relative position of the adjustable member 150 and the actuator 110 when the cutter 130 is in the fully closed position. As shown in FIG. 3B, a distal end 112 of the actuator 110 may comprise the arcuate projection 116 configured to slide through an arcuate slot 118 in the cutter 130. When the cutter 130 is in the fully closed position, the surgeon may use the drill bit 108 to drill a bone hole having a first diameter (e.g., 3.5 mm). FIG. 3C illustrates the relative position of the adjustable member 150 and actuator 110 when the cutter 130 is in an open position. As shown in FIG. 3D, the surgeon may rotate the adjustable member 150 to extend the cutter 130 from the shaft 102 to drill or over drill a bone hole having a second diameter (e.g., 10 mm). Upon rotation of the adjustable member 150, as the actuator 110 is displaced distally relative to the shaft 102, the arcuate projection 116 may slide through the arcuate slot 118, causing the cutter 130 to pivot from the closed position to an extended position. After drilling or over drilling the bone hole at the desired second diameter, either in an antegrade or retrograde direction, the surgeon may further rotate the adjustable member 150 to drill or over drill any number of additional bone holes at desired diameters (e.g., between 3.5 mm and 12 mm). When the drilling operation is finished, the surgeon may rotate the adjustable member 150 back to the 3.5 mm position. As the actuator 110 moves proximally relative to the shaft 102, the arcuate projection 116 retreats through the arcuate slot 118, pulling the cutter 130 back to the fully closed position. The device 100 then may be safely removed from the patient.

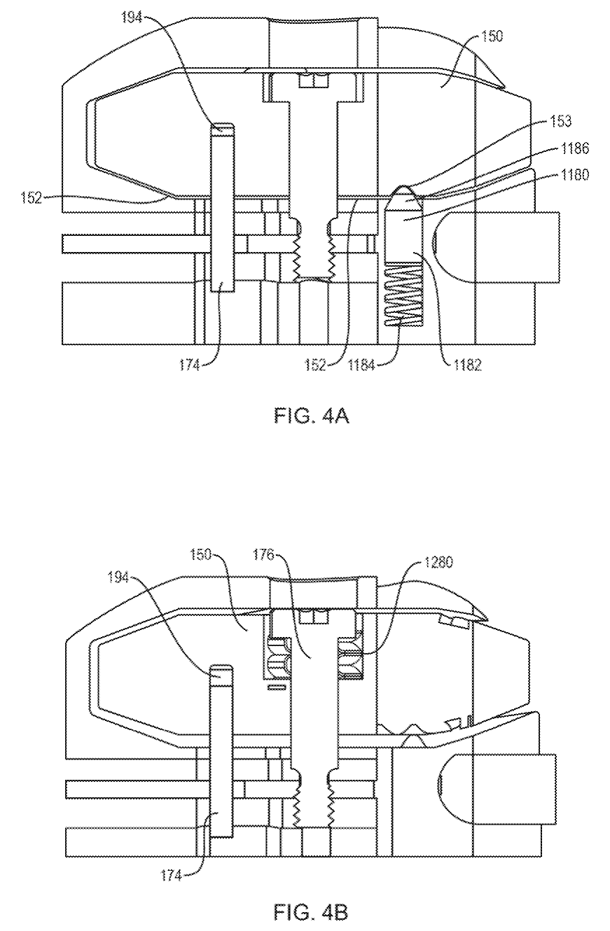

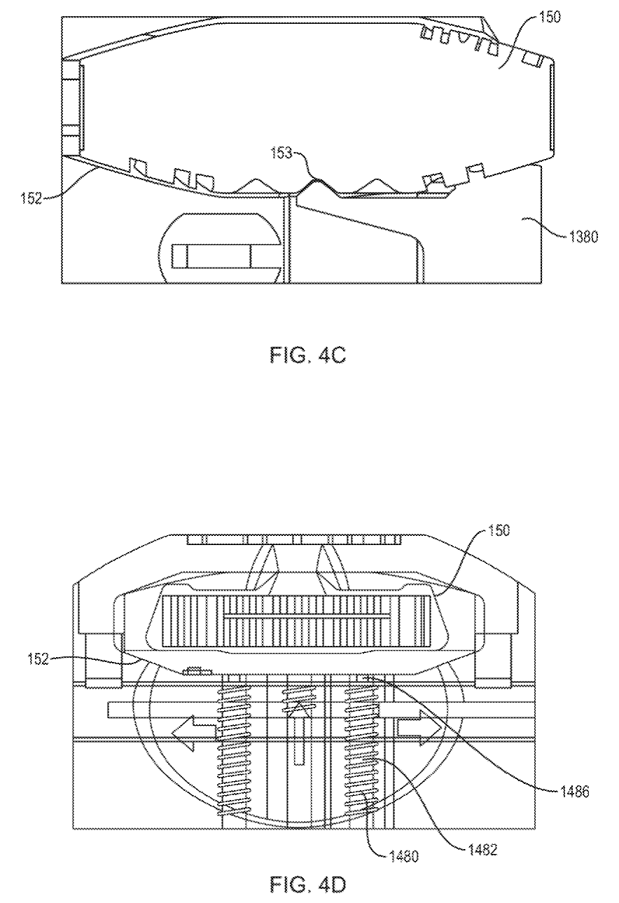

FIGS. 4A-4D illustrates various examples of the retainer 180 for engaging the adjustable member 150 to hold the adjustable member 150 in a desired rotational position.

As shown in FIG. 4A, the retainer 180 may be a spring plunger 1180. The spring plunger 1180 may include a plunger body 1182 and a spring 1184. The plunger body 1182 may comprise a tip 1186 biased into engagement with the sidewall 152 of the adjustable member 150 by the spring 1184. The tip 1186 may be sized and shaped to engage any of the recesses 153 formed in the sidewall 152. Rotation of the adjustable member 150 may reposition the tip 1186 from one recess 153 on the sidewall 152 to another recess 153 to hold the adjustable member 150 in the selected rotational orientation.

As shown in FIG. 4B, the retainer 180 may be a wave spring 1280. As shown in FIG. 4C, the retainer 180 may be a flex beam 1380. As shown in FIG. 4D, the retainer 180 may be a ball plunger 1480. The ball plunger 1480 may include a plunger body 1482 housing an internal spring (not shown). The plunger body 1482 may comprise a ball 1486 configured to be biased into engagement with the sidewall 152 of the adjustable member 150 by the internal spring. The ball 1486 may be sized and shaped to engage any of the recesses 153 formed in the sidewall 152. Rotation of the adjustable member 150 may reposition the ball 1486 from one recess 153 of the sidewall 152 to another recess 153 to hold the adjustable member 150 in the selected rotational orientation.

While the disclosure particularly shows and describes preferred embodiments, those skilled in the art will understand that various changes in form and details may exist without departing from the spirit and scope of the present application as defined by the appended claims. The scope of this present application intends to cover such variations. As such, the foregoing description of embodiments of the present application does not intend to limit the full scope conveyed by the appended claims.

What is claimed is:

1. A surgical drilling device comprising:
a shaft extending through a handle and having a proximal end, a distal end, and a longitudinal axis extending therebetween, the distal end of the shaft comprising a drill bit configured to drill a bone hole having a first diameter;
a cutter disposed within the shaft, the cutter configured to move between a closed position, in which the cutter is fully contained within the shaft, and an extended position, in which the cutter at least partially extends from a sidewall of the shaft, the cutter configured to drill a bone hole having a second diameter different from the first diameter;
an adjustable member at least partially arranged within the handle; and
an actuator disposed within the shaft and operatively coupled to both the adjustable member and the cutter, a distal end of the actuator comprising an arcuate projection configured to engage an arcuate slot in the cutter;
wherein incremental movement of the adjustable member causes a corresponding incremental degree of extension of the cutter between the closed position and the extended position to drill the bone hole having the second diameter.

2. The surgical drilling device of claim 1, wherein the first diameter is 3.5 mm.

3. The surgical drilling device of claim 1, wherein the second diameter is between 4 mm and 12 mm.

4. The surgical drilling device of claim 1, wherein the adjustable member is a rotatable dial.

5. The surgical drilling device of claim 4, wherein the rotatable dial is configured to rotate in a direction of the longitudinal axis.

6. The surgical drilling device of claim 4, further comprising a retainer configured to hold the rotatable dial in a desired rotational position.

7. The surgical drilling device of claim 6, wherein the retainer is one of a spring plunger, a wave spring, a ball plunger, and a flex beam.

8. The surgical drilling device of claim 4, wherein the rotatable dial comprises a central opening and a cam track arranged at least partially around the central opening, and wherein the cam track has a spiral formation such that a distance between a position of the cam track and the central opening varies radially.

9. The surgical drilling device of claim 8, wherein the actuator is operatively coupled to the cam track of the adjustable member by a sliding pin moveable through a slot defined in the actuator.

10. The surgical drilling device of claim 9, wherein the sliding pin is configured to travel along the cam track as a user rotates the adjustable member.

11. The surgical drilling device of claim 1, wherein the cutter is configured to extend through a slot defined in the sidewall of the shaft.

12. The surgical drilling device of claim 1, wherein an outer surface of the shaft comprises visual scale markings to indicate a penetration depth of the surgical drilling device into a bone.

13. The surgical drilling device of claim 1, wherein the cutter is pivotably connected to the distal end of the shaft by a single pivot pin.

14. The surgical drilling device of claim 1, wherein the adjustable member comprises a plurality of visual scale markings corresponding to either of the first or the second diameter.

15. The surgical drilling device of claim 1, wherein the cutter is configured to drill the bone hole having the second diameter in both an antegrade and a retrograde direction.

16. A method of drilling a bone hole, the method comprising:
placing a surgical drilling device adjacent a bone, the surgical drilling device comprising:
a shaft extending through a handle and having a proximal end, a distal end, and a longitudinal axis extending therebetween, the distal end of the shaft comprising a drill bit;
a cutter disposed within the shaft, the cutter configured to move between a closed position, in which the cutter is fully contained within the shaft, and an extended position, in which the cutter at least partially extends from a sidewall of the shaft;
an adjustable member at least partially arranged within the handle; and
an actuator disposed within the shaft and operatively coupled to both the adjustable member and the cutter, a distal end of the actuator comprising an arcuate projection configured to engage an arcuate slot in the cutter;
using the drill bit, drilling a bone hole having a first diameter;
moving the adjustable member to cause the cutter to move to the extended position; and
using the cutter, drilling a bone hole having a second diameter different from the first diameter;
wherein incremental movement of the adjustable member causes a corresponding incremental degree of extension of the cutter between the closed position and the extended position to drill the bone hole having the second diameter.

17. The method of claim 16, wherein the first diameter is 3.5 mm.

18. The method of claim 16, wherein the second diameter is between 4 mm and 12 mm.

19. The method of claim 16, wherein moving the adjustable member comprises rotating the adjustable member in a direction of the longitudinal axis.

20. The method of claim 16, wherein drilling the bone hole having the second diameter comprises drilling in either an antegrade or a retrograde direction.
